(19) United States
(12) Patent Application Publication
(10) Pub. No.: US 2025/0312614 A1
(43) Pub. Date: Oct. 9, 2025

(54) Methods and Systems for Hands-free Light Therapy Using Removable Handheld Light-Emitting Therapeutic Devices

(71) Applicant: Erchonia Corporation, LLC, Fountain Inn, SC (US)

(72) Inventors: Travis Sammons, Fountain Inn, SC (US); Jeremy Tucek, Fountain Inn, SC (US)

(73) Assignee: Erchonia Corporation, LLC, Fountain Inn, SC (US)

(21) Appl. No.: 19/095,652

(22) Filed: Mar. 31, 2025

Related U.S. Application Data
(60) Provisional application No. 63/631,880, filed on Apr. 9, 2024.

(51) Int. Cl. A61N 5/06; A61B 18/00

(52) U.S. Cl. CPC A61N 5/06; A61B 2018/00196; A61N 2005/0632; A61N 2005/0642; A61N 2005/0666

(57) ABSTRACT

Devices and methods to make it visually easy to center the light output of a light therapy device over a desired treatment area and ensure the light energy is applied uniformly over the desired treatment area. A mechanical linkage drives two light beams in opposite reciprocating motions such that each light beam scans back and forth a given distance, in opposite directions, over a desired treatment area on a patient. When the light emissions intersect periodically, the intersection indicates the center of the emitted light pattern. In a preferred embodiment, the light beams are lines and the lines remain parallel to each other. The mechanical linkage includes a cam system and a gear system.

### CROSS-REFERENCE TO RELATED APPLICATIONS

This application claims the benefit of co-pending U.S. Provisional Application No. 63/631,880 filed Apr. 9, 2024.

### FIELD OF INVENTION

This invention relates generally to light therapy devices and methods of using them. This invention relates more particularly to a light therapy treatment device and method that automatically scans light energy in a manner to enable proper placement of the light energy over a desired treatment area and to ensure the light energy is evenly applied over the desired treatment area.

### BACKGROUND

Light therapy has been shown through numerous clinical studies and regulatory clearances to be a safe and effective, simple, non-invasive and side-effect free alternative to medication and surgical procedures for the reduction of symptoms in a variety of conditions. This therapy reduces edema, improves wound healing, and relieves pain of various etiologies. It is also used in the treatment and repair of injured muscles and tendons. When the light therapy uses lasers for the light energy, the therapy is known as low-level laser therapy (“LLLT”). Application of LLLT has been shown to have the potential to alter cellular metabolism to produce a beneficial clinical effect such as fat reduction and improved cognitive function.

Subsequently, light therapy devices of various configurations have been developed to treat patients. The devices can be broadly characterized into two groups: those that emit a stationary beam of light and those that emit a scanning beam of light. For devices that emit a stationary beam of light, to cover the whole treatment area the device operator holds the device and moves his hand or arm in a back-and-forth motion to sweep the line of emitted energy across a targeted body part, thereby treating a certain area of the patient's body. This treatment method requires that the operator move his hand or arm repeatedly, which can become tiring during a long treatment or if multiple treatments are applied in series. Due to inconsistency inherent in human motion, the amplitude of each sweep varies from sweep-to-sweep, causing uncertainty in the dose of light energy applied to the target body part. Having to hold the light therapy device during treatment also prevents the operator from performing other functions during treatment.

To address those issues, light therapy devices that emit a light beam that automatically scans across a treatment area have been developed. For example, in U.S. Pat. No. 7,118,588, a scanning mechanism deflects or reflects light energy after it is emitted from the light energy source to form various shapes which are projected onto the patient. U.S. Pat. No. 7,947,067 discloses a device that emits a line of light energy that is rotated in a 360° circle to form a circular beam spot on the patient. U.S. Pat. No. 10,857,378 discloses a device that emits a line of light energy that is automatically swept up and down. U.S. Pat. Nos. 7,118,588, 7,947,067, and 10,857,378 are herein incorporated by reference.

One disadvantage of the automatic scanning mechanisms known to date is that the light may not impinge the desired body part consistently. For example, for hands-free devices, it may be difficult to determine where to position the patient to align the emitted energy over the desired treatment area. In addition, if the scan of light energy overlaps with another scan of light energy, one area of the desired treatment may receive more or less energy than another.

It would be desirable would be desirable to ensure light energy is applied evenly across a desired treatment area.

### SUMMARY OF THE INVENTION

The present invention uses a mechanical linkage to drive light emitted from two light sources in opposite reciprocating motions such that each emitted light beam scans back and forth a given distance, in opposite directions, over a desired treatment area on a patient. The mechanical linkage includes a cam system and a gear system.

When the light emitted from the first light source and the light emitted from the second light source travel across the same treatment area, the intersection of the light beams is at the center of the treatment area and the energy dose applied across the treatment area is evenly applied across the treatment area. When the light emissions intersect periodically, the intersection indicates the center of the emitted light pattern, which makes it visually easy to center the light output over the desired treatment area. In a preferred embodiment, the light energy of both light sources is emitted as a line and the lines remain parallel to each other.

### BRIEF DESCRIPTION OF THE DRAWINGS

FIG. 1 is side view of a patient being treated with a handheld version of the present device.

FIGS. 2A-D illustrate the emitted light energy as the cam system drives the light to scan in opposite directions.

FIG. 3A is a bottom view of a first linkage system in which cams are attached to the light sources, showing a cam position when the light beams are intersecting each other.

FIG. 3B is a bottom view of the first linkage system in which the cams are attached to the light sources, showing a cam position when the light beams are uncrossed.

FIG. 4A is a perspective view of a second linkage system in which the cams are attached to the light sources, showing a cam position when the light beams are intersecting each other.

FIG. 4B is a perspective view of the second linkage system in which the cams are attached to the light sources, showing a cam position when the light beams are uncrossed.

FIG. 5A is a top view of a third linkage system in which a mirror is attached to each follower and light from stationary light sources is reflected off the mirrors, such that the light beams are intersecting each other.

FIG. 5B is a top view of the third linkage system in which a mirror is attached to each follower and light from stationary light sources is reflected off the mirrors, such that the light beams are uncrossed.

FIG. 6A is a top view of a fourth linkage system in which a mirror is attached to each follower and light from stationary light sources is reflected off the mirrors, such that the light beams are intersecting each other.

FIG. 6B is a top view of the fourth linkage system in which a mirror is attached to each follower and light from stationary light sources is reflected off the mirrors, such that the light beams are uncrossed.

FIG. 7A is a top view of a fifth linkage system in which a gear is attached to each light source, showing a gear position when the light beams are intersecting each other.

FIG. 7B is a top view of the fifth linkage system in which a gear is attached to each light source, showing a gear position when the light beams are uncrossed.

FIG. 8A is a top view of a sixth linkage system in which a mirror is attached to each gear and light from stationary light sources is reflected off the mirrors, such that the light beams are intersecting each other.

FIG. 8B is a top view of the sixth linkage system in which a mirror is attached to each gear and light from stationary light sources is reflected off the mirrors, such that the light beams are uncrossed.

FIG. 9 is a partial side view of the gears in the sixth linkage system.

FIG. 10A illustrates the dosage applied to a treatment area with a uniform dose across the treatment area.

FIG. 10B illustrates the dosage applied to a treatment area with a non-uniform dose across the treatment area.

### DETAILED DESCRIPTION OF THE INVENTION

At least two light sources are mechanically linked such that they are driven in opposite reciprocating motions. Each light source scans back and forth a given distance, in opposite directions, over a desired treatment area on a patient. When the light emissions intersect periodically, the intersection indicates the center of the emitted light pattern, which makes it visually easy to center the light output over the desired treatment area. When the light emitted from the first light source and the light emitted from the second light source overlay each other on the treatment area, the intersection of the light beams is at the center of the treatment area, so that the energy dose applied across the treatment area is evenly applied to the desired treatment area. Once the light is centered over the desired treatment area, the light therapy is applied for a desired duration specific to a desired therapeutic treatment. With this approach, the inconsistency inherent in human motion is eliminated and the dose of light energy is consistently applied to the target treatment area.

In a preferred embodiment, the light energy from each light source is emitted as a line. Line-generating light therapy devices are known in the art, such as the ones disclosed in U.S. Pat. No. 6,746,473, which is incorporated herein by reference in its entirety. One embodiment includes a collimating lens and a line generating prism disposed in serial relation to the light energy source to receive and transform the generated beam of light energy into the line of light energy. In another embodiment a rod lens transforms the generated beam of light energy into the line of light energy. Alternatively, a suitable electrical or mechanical arrangement could be used to shape the light energy instead of optical elements. In the preferred embodiments, the emitted lines are parallel to each other, but alternatively the lines may be perpendicular or otherwise not parallel to each other.

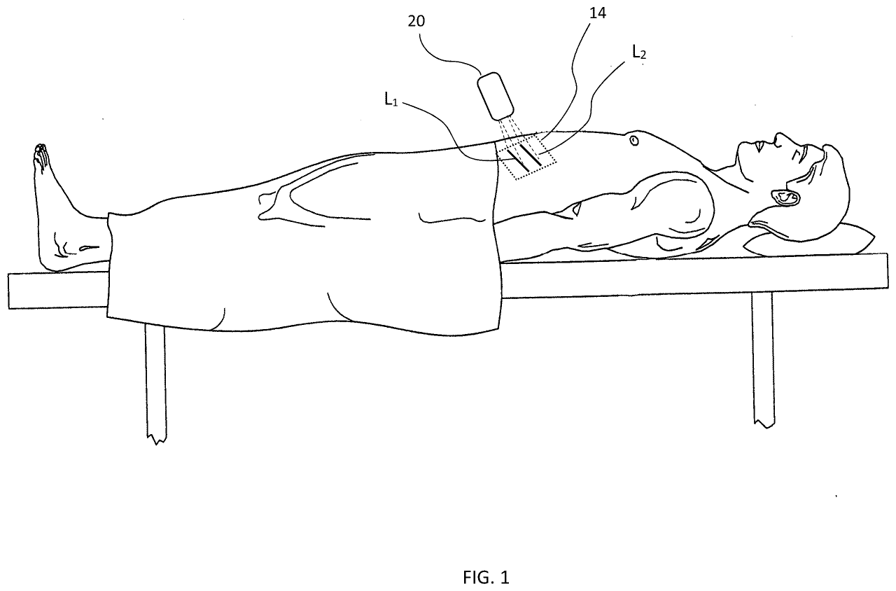

The reciprocating light-emitting devices may be housed in a standalone light therapy device or hand-held therapy devices. FIG. 1 shows a patient being treated with a handheld 20 version of a light therapy device with reciprocating beams that cross. The handheld 20 emits two beams of light energy that form two lines L₁, L₂ when they impinge the patient. The treatment area is indicated by the area inside rectangle 14.

Various configurations of a mechanical linkage are employed to drive the reciprocating motion, such as cams, gears, screws, and rack-and-pinion systems. Alternatively, an electrical or magnetic arrangement could be used to drive the reciprocating motion of the light sources.

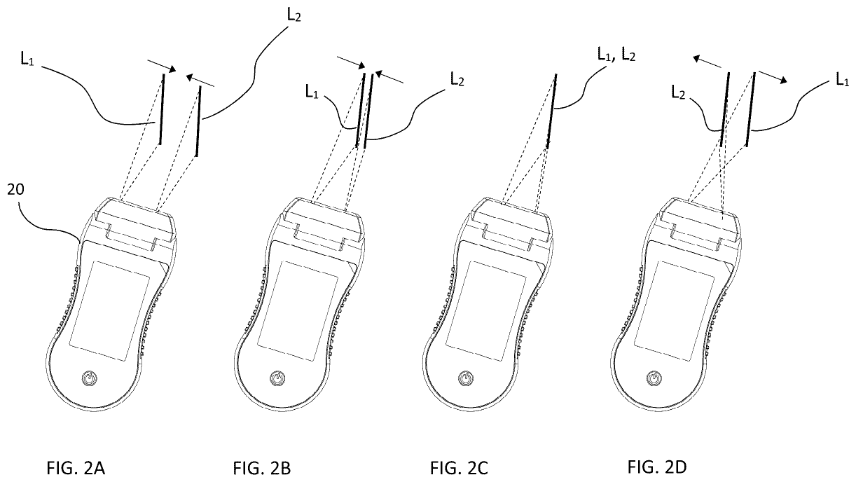

FIGS. 2A-D illustrate light energy emitted from the handheld device 20 as the linkage system drives the light to scan in opposite directions. FIG. 2A shows the light beams L₁, L₂ emitted from the handheld device 20 in an initial position. FIG. 2B shows the light beams L₁, L₂ emitted from the handheld device 20 after the light sources are moved by the linkage system to cause the beams to get closer. The lines of light remain parallel throughout. FIG. 2C shows the light beams L₁, L₂ crossing through each other and forming a single line, which is in the center of the emitted light pattern. FIG. 2D shows the light beams L₁, L₂ emitted from the handheld device 20 after they have crossed and moving away from each other. The linkage movement repeats to move the beams closer, then apart, then closer, etc., forming a scanned treatment area.

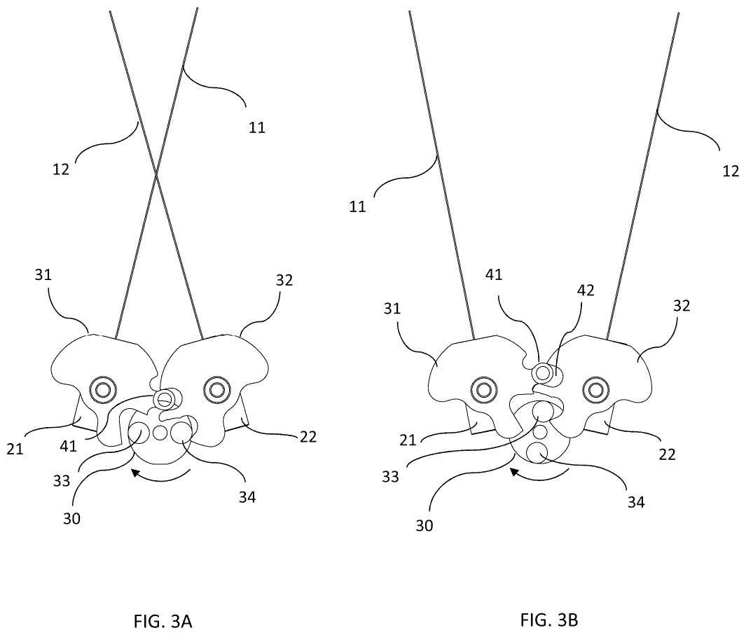

FIGS. 3A-B illustrate a first embodiment of the cam system using a round cam 30 with two posts 33, 34 driving irregularly-shaped followers 31, 32. At least one light source 21 is attached to the first irregularly-shaped follower 31 and at least one light source 22 is attached to the second irregularly-shaped follower 32. The light sources emit lines of light L₁, L₂. When the cam 30 rotates clockwise, one of the posts 33, 34 contacts the first irregularly-shaped follower 31 and rotates it a given distance in a counterclockwise direction. The first irregularly-shaped follower 31 has an extension 41 that mates with a recess 42 in the second irregularly-shaped follower 32 such that when the first irregularly-shaped follower 31 rotates counterclockwise, it rotates the second irregularly-shaped follower 32 the same given distance in a clockwise direction. Thus with every 180 degree rotation of the cam 30, a post 33 or 34 drives the first irregularly-shaped follower 31 in one direction a given distance, which in turn drives the second irregularly-shaped follower 32 in the opposite direction the given distance. As a result, with every 180 degree rotation of the cam 30, the light emitted from the light sources scans in opposite directions delineating a treatment area 14. Because the distances rotated are the same for each of the cams, the light sources move the same distance, albeit in different directions. The lines of light remain parallel throughout. Thus, when the lines L₁, L₂ pass through each other, they align for an instant to form a single line I at the center of the treatment area 14.

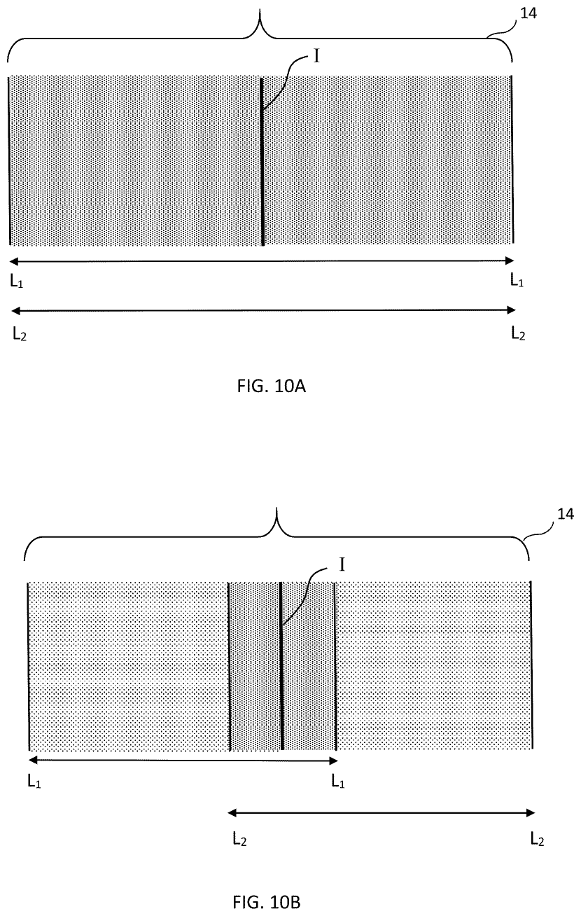

When the light emitted from the first light source and the light emitted from the second light source travel the same distance across the same treatment area, overlaying a given treatment area, the intersection of the light beams is at the center of the treatment area, and the energy dose applied across the treatment area is uniformly applied across the treatment area. See FIG. 10A. Contrast that with FIG. 10B, in which the light emitted from the first light source and the light emitted from the second light source travel the same distance, but they are not both centered across the same treatment area. The intersection of the light beams at line I is at the center of the treatment area, but the energy dose applied across the treatment area is not applied uniformly across the treatment area 14.

In some embodiments more than one light source is attached to each of the followers 31, 32. The additional light sources may provide the same or a different wavelength or power level as light sources 21, 22. The additional light sources may emit additional separate lines of light L₃. . . Ln, or the lines emitted from the additional light sources may overlap lines L₁, L₂ so that only lines L₁, L₂ appear visually. The additional light sources may be used in place of or simultaneously with light sources 21, 22 or in alternating time periods with light sources 21, 22.

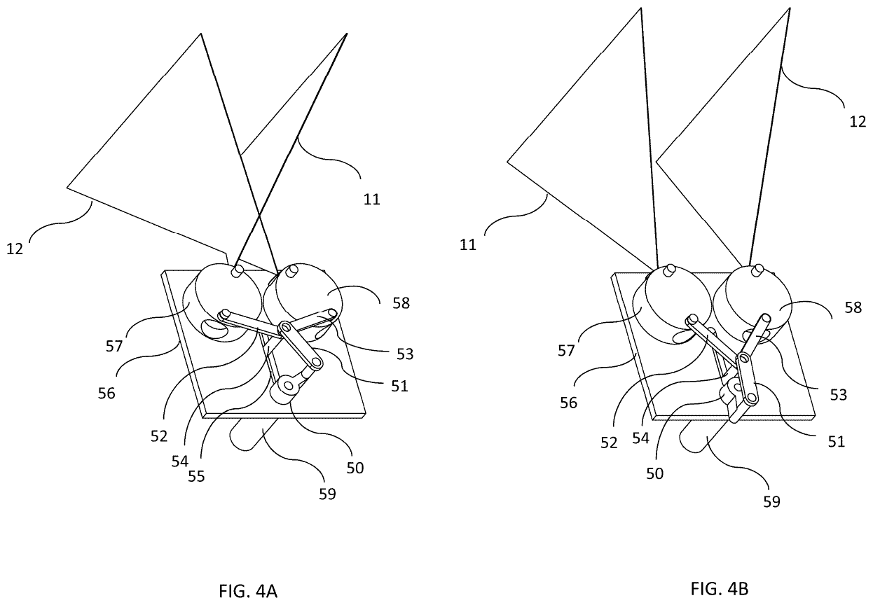

FIGS. 4A-B illustrate a second embodiment of the linkage system. At least two light sources are housed in each cylindrical housing 57, 58, each of which is disposed on a base plate 56. A round cam 50 rests on the base plate 56 and is connected to a first end of a first pushrod 51 at the perimeter of the cam 50 such that the first end of the first pushrod 51 travels in an eccentric orbit when the cam 50 is rotated. The driver is a motor 59 which, in the embodiment shown in FIGS. 4A-B, is below the base plate 56. The first pushrod 51 is connected at its second end to both a second pushrod 52 and a third pushrod 53. A guide pin 54 is attached to the first pushrod 51 at its second end, which is also the intersection of the first, second and third pushrods. The guide pin 54 slides in a guide channel 55 in a base plate 56 to keep the pushrods in alignment relative to the light sources and cam 50.

The second pushrod 52 is connected to the first light source 57 housing at its perimeter. The third pushrod 53 is connected to the second light source 58 housing at its perimeter. When the cam 50 is rotated, the first pushrod 51 drives both the second pushrod 52 and the third pushrod 53 at the same time but in opposite directions, forcing the first light source 57 and the second light source 58 to move in opposite directions. Thus with every 180 degree rotation of the cam 50, the first light source 57 is driven in one direction and the second light source 58 is driven in the opposite direction. As a result, with every 180 degree rotation of the cam 50, the light emitted from the light sources scans in opposite directions delineating a treatment area 14. The lines of light remain parallel throughout. When the lines L₁, L₂ pass through each other, they align for an instant to form a single line at the center of the treatment area 14.

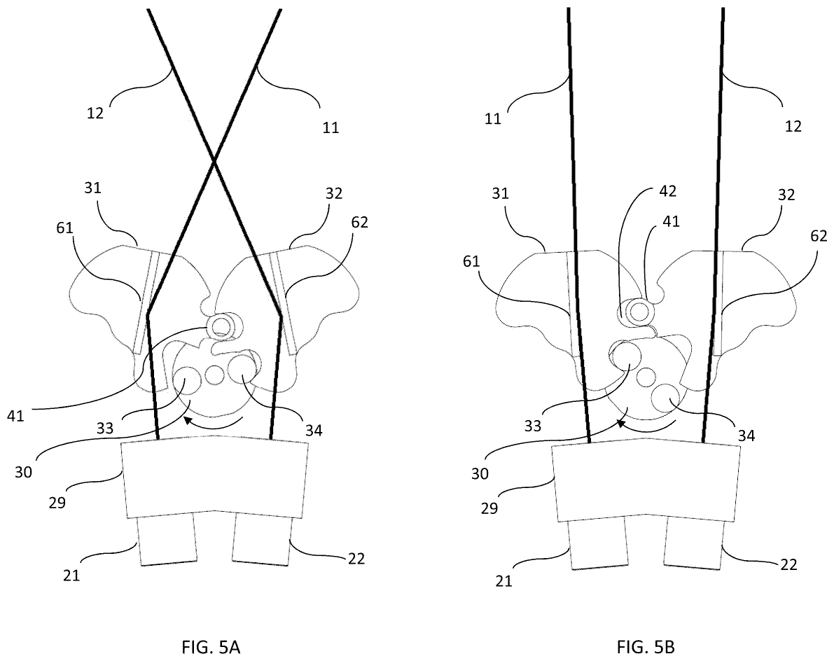

FIGS. 5A-B illustrate a third embodiment of the linkage system, in which a mirror 61, 62 is attached to each irregularly-shaped follower 31, 32. The light sources are stationary relative to the followers, and light beams from stationary light sources 21, 22 is reflected off the mirrors 61, 62. The stationary light sources are shown in a housing 29. The light sources 21, 22 emit lines of light L₁, L₂. Similar to the motion in the first embodiment, when the cam 30 rotates clockwise, one of the posts 33, 34 contacts the first irregularly-shaped follower 31 and rotates it a given distance in a counterclockwise direction. The first irregularly-shaped follower 31 has an extension 41 that mates with a recess 42 in the second irregularly-shaped follower 32 such that when the first irregularly-shaped follower 31 rotates counterclockwise, it rotates the second irregularly-shaped follower 32 the same given distance in a clockwise direction. Thus with every 180 degree rotation of the cam 30, a post 33 or 34 drives the first irregularly-shaped follower 31 in one direction a given distance, which in turn drives the second irregularly-shaped follower 32 in the opposite direction the given distance.

In contrast to the first embodiment, in the third embodiment the light sources 21, 22 are stationary relative to the followers. To achieve the reciprocating light beams 11, 12, the light emitted from each of the light sources 21, 22 strikes a mirror 61, 62 attached to each irregularly-shaped follower 31, 32. As a result, with every 180 degree rotation, the light emitted from the stationary light sources 11, 12 is reflected off the mirrors 61, 62 and scans in opposite directions delineating a treatment area 14. Because the distances rotated are the same for each of the cams, the light beams move the same distance, albeit in different directions. The lines of light remain parallel throughout. Thus, when the lines L1, L2 pass through each other, they align for an instant to form a single line I at the center of the treatment area 14.

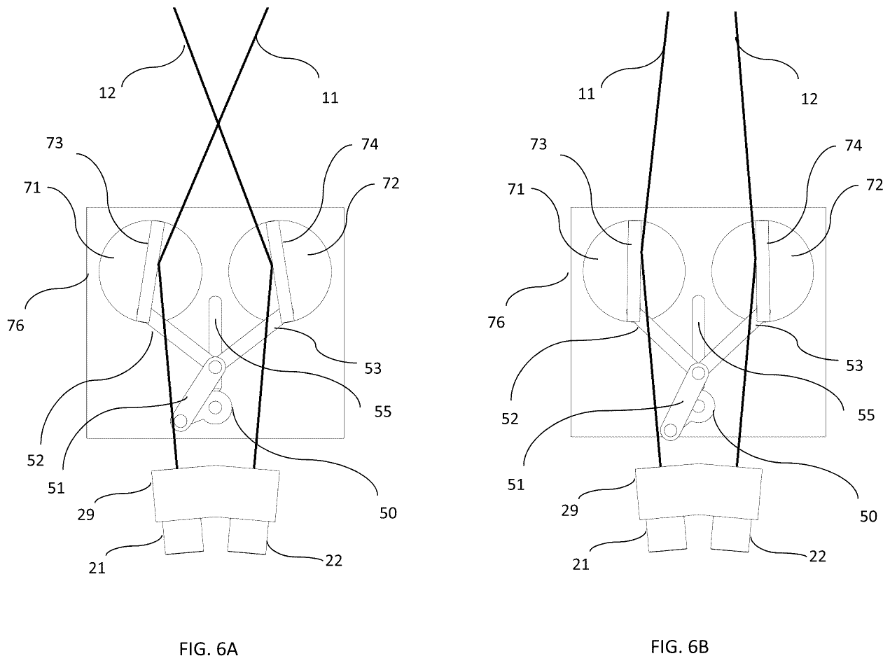

FIGS. 6A-B illustrate a fourth embodiment of the linkage system. Two mirror followers 71, 72 are disposed on a base plate 56. A mirror 73, 74 is disposed on each mirror follower 71, 72. A round cam 50 rests on the base plate 56 and is connected to a first end of a first pushrod 51 at the perimeter of the cam 50 such that the first end of the first pushrod 51 travels in an eccentric orbit when the cam 50 is rotated. The driver is a motor 59 which, in the embodiment shown in FIGS. 6A-B, is below the base plate 56. The first pushrod 51 is connected at its second end to both a second pushrod 52 and a third pushrod 53. A guide pin 54 is attached to the first pushrod 51 at its second end, which is also the intersection of the first, second and third pushrods. The guide pin 54 slides in a guide channel 55 in a base plate 56 to keep the pushrods in alignment relative to the light sources and cam 50.

When the cam 50 is rotated, the first pushrod 51 drives both the second pushrod 52 and the third pushrod 53 at the same time but in opposite directions, forcing the first mirror follower 71 and the second mirror follower 72 to move in opposite directions. Two stationary light sources 21, 22 are shown in a housing 29. The light sources 21, 22 emit lines of light L₁, L₂ that strike the mirrors 73, 74. Thus light from stationary light sources 21, 22 is reflected off the mirrors. As a result, with every 180 degree rotation of the cam 50, the light emitted from the light sources scans in opposite directions delineating a treatment area 14. The lines of light remain parallel throughout. When the lines L₁, L₂ pass through each other, they align for an instant to form a single line at the center of the treatment area 14.

The cams or drive gear are preferably driven by a battery-powered motor, but may also be driven by other means, such as a mains-powered motor, a manual mechanism, a wind-up mechanism, or a pneumatic mechanism. Typically the cam 30 or 50 continuously rotates 360 degrees in one direction, either clockwise or counterclockwise. However in some embodiments the cam 30, the cam 50, or the drive gear 83 may rotate an amount less than 360 degrees in one direction, then reverse direction and rotate that amount in the opposite direction, switching back and forth in a repetitive cycle. This ability enables the size of the treatment area to be adjusted by changing the degree of rotation in each direction. A smaller degree of rotation forms a smaller treatment area. Cam systems with more than one cam may be used.

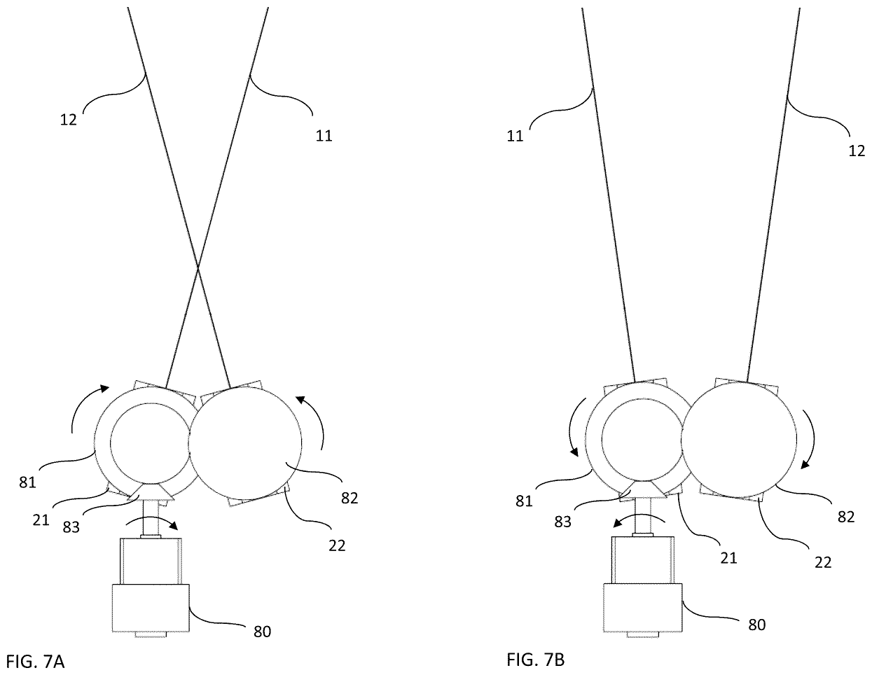

FIGS. 7A-B illustrate a fifth embodiment of the linkage system. This embodiment drives the reciprocating light beams using a motor connected to gears instead of a cam system. At least one light source 21 is attached to a first gear 81 and at least one light source 22 is attached to a second gear 82. The first gear 81 has teeth (not shown) on the top that mate with teeth (not shown) on the bottom of the second gear 82. The light sources emit lines of light L₁, L₂.

A motor 80 is connected to a drive shaft which has a drive gear 83 that mates with the first gear 81. When the drive gear 83 rotates, it causes the first gear 81 to rotate in one direction and the mated second gear 82 to rotate in the opposite direction. When the gears 81, 82 rotate, this cause the light sources 21, 22 to rotate in the same direction as the gears they are attached to, causing the emitted light to scan. FIG. 7A shows the drive gear 83 causing the first light source 21 to rotate in a clockwise direction and the second light source 22 to rotate in a counterclockwise direction. FIG. 7B shows the drive gear 83 causing the first light source 21 to rotate in a counterclockwise direction and the second light source 22 to rotate in a clockwise direction. As a result, with every rotation of the drive gear 83, the light emitted from the light sources scans in opposite directions delineating a treatment area 14. Because the distances rotated are the same for each of the cams, the light sources move the same distance, albeit in different directions. The lines of light remain parallel throughout. Thus, when the lines L1, L2 pass through each other, they align for an instant to form a single line I at the center of the treatment area 14.

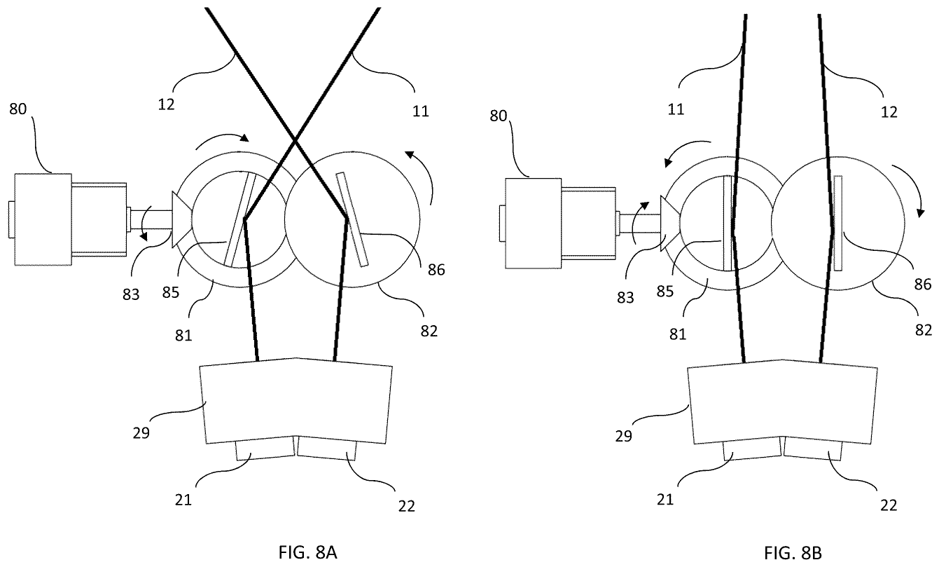

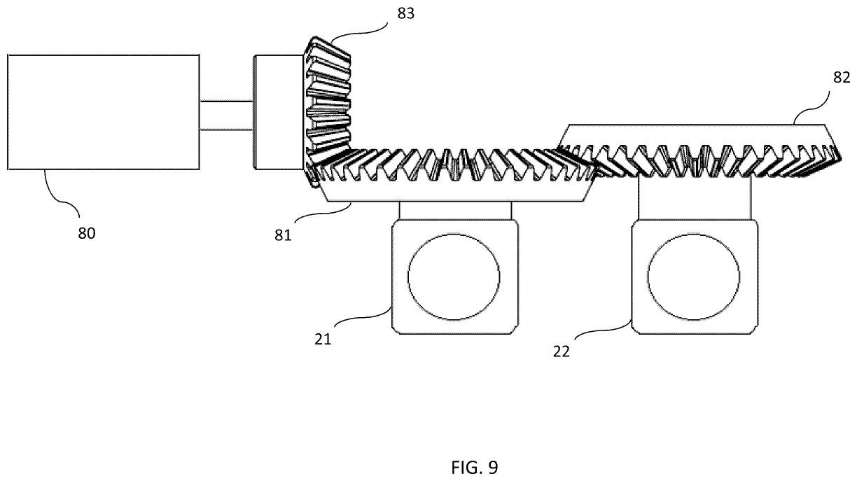

FIGS. 8A-B and FIG. 9 illustrate a sixth embodiment of the linkage system. This embodiment drives the reciprocating light beams using a motor connected to gears, but instead of having the light sources 21, 22 attached to the gears as in the fifth embodiment above, the light sources are stationary relative to the gears. The light sources emit lines of light L₁, L₂.

A mirror 85, 86 is attached to each gear 81, 82. As shown in FIG. 9 first gear 81 has teeth on the top that mate with teeth on the bottom of the second gear 82. A motor 80 is connected to a drive shaft which has a drive gear 83 that mates with the first gear 81. When the drive gear 83 rotates, it causes the first gear 81 to rotate in one direction and the mated second gear 82 to rotate in the opposite direction. When the gears 81, 82 rotate, this cause the mirrors 85, 86 to rotate in the same direction as the gears they are attached to. FIG. 8A shows the drive gear 83 causing the first light mirror 85 to rotate in a clockwise direction and the second mirror 86 to rotate in a counterclockwise direction. FIG. 8B shows the drive gear 83 causing the first mirror 85 to rotate in a counterclockwise direction and the second mirror 86 to rotate in a clockwise direction. The light emitted from each of the stationary light sources 21, 22 strikes the mirror 85, 86 attached to each gear 81, 82. As a result, with every 180 degree rotation, the light emitted from the stationary light sources 11, 12 is reflected off the mirrors 85, 86 and scans in opposite directions delineating a treatment area 14. Because the distances rotated are the same for each of the cams, the light beams move the same distance, albeit in different directions. The lines of light remain parallel throughout. Thus, when the lines L1, L2 pass through each other, they align for an instant to form a single line I at the center of the treatment area 14.

As a result, with every rotation of the drive gear 83, the light emitted from the light sources scans in opposite directions delineating a treatment area 14. Because the distances rotated are the same for each of the cams, the light sources move the same distance, albeit in different directions. The lines of light remain parallel throughout. Thus, when the lines L1, L2 pass through each other, they align for an instant to form a single line I at the center of the treatment area 14.

Light therapy treatment uses wavelengths in the visible range, about 400 nm to 1000 nm, depending on the desired treatment. In some embodiments only a single wavelength is used, for example 635 nm. The source of the light energy is preferably semiconductor laser diodes, but may also be from light-emitting diodes (LEDs). Commercial semiconductor laser diodes have a spread of ±10 nm from nominal so, for a given desired wavelength, the light applied is within the spread from nominal. In contrast, LEDs have a wider spread of wavelengths covering a desired color, for example red light. In the preferred embodiment each light source emits a different color, but in some embodiments the same color light energy is emitted by both light sources.

The light energy is applied with a pulse frequency or frequencies from 0 to 100,000 Hz. The applied light energy does not create heat, for example by using from conventional laser diode emitters of less than 1 W, often emitting less than 7.5 mW, or from super pulse lights over 1 W. Consequently, the tissue impinged by the light is not heated and is not damaged.

In practice, the patient is seated or lying down on a table, depending on the best position to access the area to be treated. The light therapy device is turned on. A first line of light energy is emitted at a first initial position and a second line of light energy is emitted at a second initial position. The lines scan in opposite reciprocating motions, towards and away from each other. The light scans at such a high frequency that the unaided eye cannot actually see the evolution of the light starting to scan, and instead sees two bright lines of light that pass over the skin of the patient, intersecting periodically into a single line. The device is positioned so that the single line is centered over the desired area of treatment on the patient.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims and equivalents thereof.

We claim:

1. A method of light therapy applied to a desired treatment area, the method comprising:
a. emitting a first beam of light energy at a first initial position;
b. emitting a second beam of light energy at a second initial position;
c. moving the first beam of light energy a given distance toward the second initial position;
d. moving the second beam of light energy the given distance toward the first initial position;
such that the first beam of light energy and the second beam of light energy intersect to form a single beam; and
e. centering the single beam over the desired treatment area.

2. The method of claim 1 further comprising:
a. moving the first beam of light energy a given distance away from the second initial position;
b. moving the second beam of light energy the given distance away from the first initial position;
such that the first beam of light energy and the second beam of light energy intersect again; and
c. repeating steps a-b of this claim 2 for a desired duration.

3. The method of claim 1 wherein the first light beam is a line and the second light beam is a line.

4. A method of light therapy applied to a desired treatment area, the method comprising:
a. emitting a first line of light from a first light source;
b. emitting a second line of light from a second light source; and
c. driving the first light source in a first reciprocating motion and driving the second light source in a second reciprocating motion in a direction opposite the first reciprocating motion, such that the first line of light energy and the second line of light energy intersect to form a single line over the desired treatment area.

5. A light therapy device comprising:
a. a first light source emitting a first light beam;
b. a second light source emitting a second light beam; and
c. a mechanical linkage that drives the first light beam in a first reciprocating motion and drives the second light beam in a second reciprocating motion in a direction opposite the first reciprocating motion, such that the first light beam periodically intersects the second light beam.

6. The light therapy device of claim 5 wherein the mechanical linkage is a cam system comprising:
a. the first light source attached to a first irregularly-shaped follower;
b. the second light source attached to a second irregularly-shaped follower; and
c. a cam with a first post and a second post wherein, when the cam is rotated, the first post drives the first irregularly-shaped follower in a first direction and
a second post drives the second irregularly-shaped follower in a direction opposite the first direction.

7. The light therapy device of claim 5 wherein the mechanical linkage is a cam system comprising:
a. a first pushrod having a first end and a second end; and
b. a cam connected to the first end of the first pushrod at the perimeter of the cam, such that when the cam is rotated, the first pushrod drives the first light source and the second light source in opposite directions.

8. The light therapy device of claim 5 wherein the mechanical linkage is a cam system comprising:
a. a first pushrod having a first end and a second end;
b. a cam connected to the first end of the first pushrod at the perimeter of the cam, such that the first end of the first pushrod travels in an eccentric orbit when the cam is rotated; wherein
c. the first pushrod is connected at its second end to both a second pushrod and a third pushrod;
d. the second pushrod is connected to the first housing at its perimeter; and
e. the third pushrod is connected to the second housing at its perimeter;
such that when the cam is rotated, the first pushrod drives both the second pushrod and the third pushrod at the same time but in opposite directions, forcing the first light source and the second light source to move in opposite directions.

9. The light therapy device of claim 5 wherein the mechanical linkage is a cam system comprising:
a. a first mirror attached to a first irregularly-shaped follower;
b. a second mirror attached to a second irregularly-shaped follower; and
c. a cam with a first post and a second post wherein, when the cam is rotated, the first post drives the first irregularly-shaped follower in a first direction and a second post drives the second irregularly-shaped follower in a direction opposite the first direction; and wherein
d. the first light source is stationary relative to the first irregularly-shaped follower and disposed in a position such that the first light beam strikes the first mirror and the second light source is stationary relative to the second irregularly-shaped follower and is disposed in a position such that the second light beam strikes the second mirror.

10. The light therapy device of claim 5 wherein the mechanical linkage is a cam system comprising:
a. a first pushrod having a first end and a second end;
b. a first cam connected to the first end of the first pushrod at the perimeter of the cam, such that the first end of the first pushrod travels in an eccentric orbit when the cam is rotated; wherein
c. the first pushrod is connected at its second end to both a second pushrod and a third pushrod;
d. the second pushrod is connected to a first mirror disposed on a first mirror follower; and
e. the third pushrod is connected to a second mirror disposed on a second mirror follower;
f. the first light source is stationary relative to the first mirror and disposed in a position such that the first light beam strikes the first mirror; and
g. the second light source is stationary relative to the second mirror and is disposed in a position such that the second light beam strikes the second mirror;
such that when the first cam is rotated, the first pushrod drives both the second pushrod and the third pushrod at the same time but in opposite directions, forcing the first light beam and the second light beam to move in opposite directions.

11. The light therapy device of claim 5 wherein the mechanical linkage is a gear system comprising:
a. the first light source attached to a first gear;
b. the second light source attached to a second gear; and
c. a motor driving a drive gear such that the drive gear drives the first gear and the second gear in opposite directions.

12. The light therapy device of claim 5 wherein the mechanical linkage is a gear system comprising:
a. a mirror attached to a first gear;
b. a mirror attached to a second gear;
c. the first light source is stationary relative to the first mirror and disposed in a position such that the first light beam strikes the first mirror;
d. the second light source is stationary relative to the second mirror and is disposed in a position such that the second light beam strikes the second mirror; and
e. a motor rotating a drive gear such that the drive gear drives the first gear and the second gear in opposite directions;
such that when the first cam is rotated, the first pushrod drives both the second pushrod and the third pushrod at the same time but in opposite directions, forcing the first light beam and the second light beam to move in opposite directions.
